(19) United States
(12) Patent Application Publication
(10) Pub. No.: US 2006/0222638 A1
(43) Pub. Date: Oct. 5, 2006

(54) Cholesterol biosynthesis pathway modulators and uses thereof

(75) Inventors: Kandiah Jeyaseelan, Donvale (AU); Arunmozhiarasi Armugam, Singapore (SG); Siaw Ching Chai, Singapore (SG); Prabhakaran Nair Ramkishen, Singapore (SG); Ponnampalam Gopalakrishnakone, Singapore (SG); Kwon Huat Benny Tan, Singapore (SG)

Correspondence Address: ROTHWELL, FIGG, ERNST & MANBECK, P.C., 1425 K STREET, N.W., SUITE 800, WASHINGTON, DC, 20005, US

(73) Assignee: National University of Singapore, Sinapore (SG)

(21) Appl. No.: 10/559,649

(22) PCT Filed: Jun. 4, 2004
(86) PCT No.: PCT/SG04/00168

§ 371 Date: Dec. 5, 2005

Related U.S. Application Data
(60) Provisional application No. 60/476,208, filed on Jun. 5, 2003.

(51) Int. Cl. C12Q 1/68; C07H 21/04; C12P 21/06; A61K 38/48; C12N 9/64

(52) U.S. Cl. 424/94.64; 435/6; 435/69.1; 435/320.1; 435/226; 435/325; 530/350; 536/23.2

(57) ABSTRACT

An isolated peptide comprising the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol. Also disclosed is a pharmaceutical composition comprising the peptide having sequence SEQ ID NO:2 or a variant, derivative and/or fragment thereof. Also disclosed is a method for treatment or prophylaxis of disorders characterised by the accumulation of cholesterol, its by-products and/or related lipid derived products, comprising administering to a subject in need at least one peptide comprising the amino acid sequence of SEQ ID NO: 2, or a variant, derivative and/or fragment thereof.

### FIELD OF THE INVENTION

The present invention relates to Cholesterol biosynthesis pathway modulators. In particular, to polypeptides or variants, derivatives and/or fragments thereof having the function of HMGCoA reductase inhibitors, phosphomevalonate inhibitors, reducing the accumulation of cholesterol in the cholesterol biosynthesis pathway and/or reducing the level of serum cholesterol. The invention also relates to methods of treatment or prophylaxis of disorders characterised by the accumulation of cholesterol, its by-product and/or related lipid derived products, comprising administering to a subject any polypeptide of the invention.

### BACKGROUND OF THE INVENTION

Cholesterol is needed for cell membrane structure and as a precursor for bile acid and steroid hormones synthesis. Cholesterol is also required for cellular signalling pathway. Nevertheless, excess cellular free cholesterol is undesirable. Elevated plasma low density lipoprotein, (LDL)-cholesterol levels and low levels of high density lipoproteins (HDL)-cholesterol are known risk factors for the development and progression of atherosclerosis and coronary heart diseases (Brunzell and Hokanson, 1999, Am J Med 1999 August 23;107(2A):16S-18S; Tribble D L, Krauss R M., Adv Intern Med, 1993;38:1-29). Several epidemiological studies have also demonstrated that elevated plasma concentrations of triglycerids and low levels of HDL are a risk factor for the development of coronary heart diseases (Gotto A M Jr., Am J Manag Care 2002 January ;Suppl:2, 4). HMG-CoA reductase (HMGR) plays an important and rate-limiting role in cholesterol biosynthesis. This enzyme is responsible for catalyzing the conversion of HMG-CoA to mevalonate in the cholesterol biosynthetic pathway and is the target of compounds that are very effective in lowering serum cholesterol.

Hyperlipidemic patients are treated with cholesterol lowering medications and particularly with inhibitors of 3-hydroxy-3-methylglutaryl coenzyme A (HMG-CoA) reductase. Statins are specific inhibitors of HMG-CoA reductase. These drugs have shown to lower the LDL-cholesterol level of patients and are the most effective and tolerated drugs currently used in the treatment of hypercholesterolemia (Brown et al, Am Heart J. 2002 December, 144(6):1036-43).

Currently there are five statins, lovastatin, simvastatin, pravastatin, fluvastatin, and atorvastatin available. These drugs lower cholesterol by slowing down the production of cholesterol and by increasing the liver's ability to remove the LDL-cholesterol present in the blood. Studies have shown that about 20 to 60 percent lowering of LDL-cholesterol levels can be brought about in patients using these drugs.

Statins are also known to reduce triglyceride levels and cause an increase in HDL-cholesterol, as well as elevate the synthesis of LDL-receptors. However, different types of statins seem to show significantly different activities, either at the same or different doses (Serajuddin et al, J Pharm Sci 1991 September;80(9):830-4). They differ not only in their mode of action but also in their potency (Cohen L H, et al., Biopharm Drug Dispos 2000 December;21(9):353-64), metabolic rate, tissue selectivity/affinity, absorption (Nezasa K, et al., Drug Metab Dispos 2002 November;30(1i):1158-63) and duration of action. Nevertheless, all the statins show lower incidence of adverse effects.

Accordingly, there is a need in the art for the availability of further and alternative drugs for reducing the level of serum cholesterol.

### SUMMARY OF THE INVENTION

The present invention addresses the problems above, and provides new cholesterol biosynthesis pathway modulators. In particular, the present invention provides polypeptides or variants, derivatives and/or fragments thereof having the function of HMGCoA reductase inhibitors, phosphomevalonate inhibitors, reducing the accumulation of cholesterol in the cholesterol biosynthesis pathway and/or reducing the level of serum cholesterol. The invention also relates to methods of treatment or prophylaxis of disorders characterised by the accumulation of cholesterol, its by-products and/or related lipid derived products, comprising administering to a subject any polypeptide of the invention.

According to a first aspect, the invention provides an isolated nucleic acid molecule selected from the group consisting of:
(a) nucleic acid molecules comprising the nucleotide sequence of SEQ ID NO:1;
(b) nucleic acid molecules encoding a peptide having the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol;
(c) nucleic acid molecules which hybridize to a nucleic acid molecule complementary to the nucleic acid molecule of (a), or (b) or fragment thereof, and which encode a peptide having the function of HMGCoA reductase inhibitor, phosphomevalonate inhibitor, reducing the accumulation of cholesterol in the cholesterol biosynthesis pathway and/or reducing the level of serum cholesterol;
(d) nucleic acid molecules comprising a nucleotide sequence having at least 40% identity with the sequence of SEQ ID NO:1 or a fragment thereof and which encode a peptide having the function of HMGCoA reductase inhibitor, phosphomevalonate inhibitor, reducing the accumulation of cholesterol in the cholesterol biosynthesis pathway and/or reducing the level of serum cholesterol.

In particular, the nucleic acid molecule (b) may be a nucleic acid molecule encoding at least a peptide having the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or SEQ ID NO:13.

The nucleic acid molecule (d) may be a nucleic acid molecule comprising a nucleotide sequence having at least 40%, 50%, 60%, 70%, 80% or 90% identity with the sequence of SEQ ID NO:1 or a fragment thereof and which encodes a peptide having the function of HMGCoA reductase inhibitor, phosphomevalonate inhibitor, reducing the accumulation of cholesterol in the cholesterol biosynthesis pathway and/or reducing the level of serum cholesterol.

The nucleic acid molecule of the invention can be a single or double stranded polynucleotide, oligonucleotide, genomic DNA, cDNA, RNA, mRNA.

The invention also provides a vector comprising at least one of the nucleic acid molecules (a), (b), (c), (d). In particular, the vector may comprise at least two from the nucleic acid molecules (a), (b), (c) and (d) are fused together.

The vector may further comprise a regulatory nucleic acid sequence, which is linked to the nucleic acid molecule encoding the polypeptide. The regulatory nucleic acid may be a prokaryotic or eukaryotic promoter.

The invention further provides a host cell comprising the vector. The host cell may be present in the form of a cell culture. The host cell may be a prokaryotic or eukaryotic cell.

In particular, the host cell is cultured to express a peptide having the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol; The host cell may also express at least a peptide having the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or SEQ ID NO:13.

According to another aspect, the invention provides an isolated peptide comprising the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol. The peptide may comprise at least one of the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13.

The peptide of the invention may be a fused peptide and comprises at least one peptide having the sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or SEQ ID NO:13.

The peptide of the invention may be isolated and/or purified from biological material, expressed from recombinant DNA, and/or prepared by chemical synthesis.

In particular, the peptide may be isolated and/or purified from a human or non-human animal species.

According to another aspect, the peptide of the invention is isolated and/or purified from the venom. Any suitable venom from any animal comprising venom can be used. The peptide can be isolated and/or purified according to any standard technology known in the art. In particular, the peptide may be obtained from steps comprising:
obtaining crude venom;
carrying out gel filtration; and
performing reverse-phase high performance liquid chromatography.

However, the method of isolating and/or obtaining the peptide of the invention may be any other method known in the art.

According to one example, the venom is from Buthus martensii Karsch, however, venom from other species may also be used. Accordingly, in particular, the peptide of the invention is isolated and/or purified from the step comprising:
obtaining crude venom of a Buthus martensii Karsch;
carrying out gel filtration;
performing reverse-phase high performance liquid chromatography.

According to another aspect, the invention provides a pharmaceutical preparation comprising the peptide of the invention. The pharmaceutical preparation optionally comprises a pharmaceutically acceptable carrier, diluent, excipient or a combination thereof.

The pharmaceutical preparation may be in the form of oral, parenteral, injection, topical, and/or implant preparation.

According to another aspect, the invention provides a method of treatment or prophylaxis of disorders characterised by the accumulation of cholesterol, its by-product and/or related lipid derived products, comprising administering to a subject in need at least one peptide comprising the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol. The method may also comprise administering at least one peptide or a fused peptide comprising the amino acid sequence of at least one of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13. Further, the method of the invention may also comprise administering a pharmaceutical composition according to the invention.

The by-product comprises bile acid. The related lipid derived products comprises HDL, LDL and/or VLDL.

The method disorders comprise hypertension, atherosclerosis, stroke, neurovascular and/or cardiovascular disorders. The peptide or the pharmaceutical preparation may be administered locally, by injection, implantation, topical administration to a tissue locus, parenterally and/or orally. Other forms of administration known in the art and suitable for the purposes of the present method are also within the scope of the present invention.

According to another aspect, the invention provides a method of treatment for cholesterol independent and pleiotropic conditions comprising administering to a subject at least one peptide comprising the amino acid sequence of SEQ ID NO: 2. The method may also comprise administering at least one peptide or a fused peptide comprising the amino acid sequence of at least one of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13.

The cholesterol independent and pleiotropic condition comprise, but are not limited to, atherosclerotic stabilization, amelioration of endothelial dysfunction, improved coronary artery compliance, prevention of plaque rupture, and/or thrombus formation.

According to another aspect, the invention provides a use of at least one peptide comprising the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol, for the preparation of a medicament for the treatment or prophylaxis of disorders characterised by the accumulation of cholesterol, its by-product and/or related lipid derived products. In particular, the by-product comprises bile acid while related lipid derived products comprises HDL, LDL and/or VLDL. The disorders comprise hypertension, atherosclerosis, stroke, neurovascular and/or cardiovascular disorders.

The medicament comprises at least one peptide or a fused peptide comprising the amino acid sequence of at least one of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13.

According to a further aspect, the medicament further comprises a pharmaceutically acceptable carrier, diluent, excipient or a combination thereof. The medicament is administered locally, by injection, implantation, topical administration to a tissue locus, parenterally and/or orally.

Another aspect of the present invention is a use of at least one peptide comprising the amino acid sequence of SEQ ID NO: 2 for the preparation of a medicament for the treatment for cholesterol independent and pleiotropic conditions. In particular, the cholesterol independent and pleiotropic condition comprise atherosclerotic stabilization, amelioration of endothelial dysfunction, improved coronary artery compliance, prevention of plaque rupture, and/or thrombus formation.

Further, the medicament comprises at least one peptide or a fused peptide comprising the amino acid sequence of at least one of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13.

### BRIEF DESCRIPTION OF THE FIGURES

FIGS. 1(A and B): HMGCoA reductase activity of JC and Simvastatin using (a) rat hepatocyte and (b) HepG2 cell enzymes. FIG. 1A: HMGCoA reductase assay, using the enzyme isolated from rat hepatocytes, using both simvasatin (10 uM) and crude JC (10 ug). The inhibitors were added to the enzyme assay. FIG. 1B: HMGCoA reductase assay for HepG2 cells treated with simvastatin (10 uM) and crude JC (10 ug). The cells were then scraped and sonicated to break the cell for proteins. The cell proteins were used as enzyme source. In both cases, the positive controls were cells without any treatment while the negative controls were assays without the enzyme

FIGS. 2(A, B, C, D): Gel filtration chromatography and RP-HPLC purifications of native JCH2 from Buthus martensii Karsch venom (a) Sephadex G-50 gel filtration chromatography of the crude venom; (b) RP-HPLC of the active fraction (13/14/15) from G-50 gel filtration on a Jupiter C18 column; (c) RP-HPLC of the active fraction (H2) from (b) on Jupiter C4 column; (d) final RP-HPLC purification of JCH2 on Jupiter C4 column from the active fraction (c).

FIG. 3: PCR based cDNA cloning was carried out. Primers were designed based on the determined N-terminal sequence of native JCH2. cDNA (SEQ ID NO:1) and deduced amino acid sequence of JCH2 (SEQ ID NO:2). The N-terminal sequence obtained from direct amino acid sequencing (SEQ ID NO:4) is underlined.

FIG. 4: Mass analysis of JCH2 was carried out on a Q-TOF mass spectrophotometer (Micromass UK Ltd). The mass spectrum was processed using Micromass MassLynx software. The mass is determined to be 16803.9 Da. The N-terminal sequencing (Edman Degradation) of JCH2 was carried out on Procise-HT protein sequencer attached to a 140C PTH analyser (Applied Biosystems, USA). The partial amino acid sequence were determined to be: DSLSPWNEGDTYYGCQRQTDEFCNKICKLH (SEQ ID NO:4).

FIGS. 5(A, B): (a) The 3D structure of JCH2 was predicted based on homology modelling facility available on: (http://www.expasy.org/swissmod/SWISS-MODEL.html) using the first approach mode model and (b) the deduced amino acid sequence of JCH2 and the synthetic peptides derived from the sequence. The numbers refer to the individual peptide fragments (peptides 1-9) as shown in Table 2. Letter H, C and E refer to helix, coil and beta strand respectively.

FIGS. 6(A, B): Cell viability (MTT) assay on HepG2 cell treated (a) 4 hours of incubation, (b) 24 hours of incubation, with various concentration of statins (10 uM each), JC (crude venom), G50 (partially purified JC; FIG. 2A) and JCH2 (pure HMGCoA reductase inhibitor from venom; FIG. 2D). Viability (Abs at 550) was plotted against protein concentration of the inhibitors.

### DETAILED DESCRIPTION OF THE INVENTION

The present invention provides new cholesterol biosynthesis pathway modulators. In particular, the present invention provides polypeptides or variants, derivatives and/or fragments thereof having the function of HMG-CoA reductase inhibitors, phosphomevalonate inhibitors, reducing the accumulation of cholesterol in the cholesterol biosynthesis pathway and/or reducing the level of serum cholesterol.

HMG-CoA (3-hydroxy-3-methylglutaryl-coenzyme A) reductase is the enzyme which catalyzes the rate limiting step of cholesterol biosynthesis. HMG-CoA reductase inhibitors, also known as statins, are molecules which inhibit the enzymatic activity of HMG-CoA reductase and have been used to treat patients suffering from hypercholesterolemia. The first such inhibitor (compactin or Mevastatin) was isolated in 1976 (Endo, A. et al., F.E. B.S. Left., 72: 323-326, 1976) and since then many other natural and chemically modified versions of Mevastatin have been identified and developed for clinical use, including Lovastatin (Mevacor), and Simvastatin (Zocor).

Additionally, several studies have shown that HMG-CoA reductase inhibitors have anti-angiogenic activity. Feleszko, W. et. al., Int. J. Cancer, 81: 560-567 (1999) reported that treatment of a mouse model of tumour-cell induced angiogenesis with a combination of TNF-alpha and lovastatin produced a significant inhibition of tumor-induced blood-vessel formation whereas treatment with either TNF-alpha or lovastatin alone showed no. angiostatic effects. Jones, M. K. et al., Am. J. Physiol., 276: G1345-GI1355 (1999) reported that mevastatin, an inhibitor of Ras activation, completely blocked the induction of VEGF (a potent angiogenic factor) expression in cultured primary endothelial cells. Kong, D. et al., Circulation, 100(18): I-39, Abstract #194 (1999) reported that simvastatin exerted potent anti-angiogenic effects independent of its cholesterol lowering effects.

Recent studies have shown that in addition to the treatment of hyperlipidemia, HMG-CoA reductase inhibitors are useful in the treatment of acne and/or skin aging (see, e.g. Breton, L. et al., U.S. Pat. No. 5,902,805); can increase nitric oxide (NO)-mediated vasodilation and blood vessel relaxation (see e.g., Liao, J. K. et al., WO 99/18952); and can help prevent a second or additional myocardial infarction (see, e.g., Behounek, B. D. et al., U.S. Pat. No. 5,674,893; Olukotun, A. Y. et al., U.S. Pat. No. 5,622,985).

The regulation of serum cholesterol in mammals and particularly primates has attracted significant attention. It is often reported that regulation of cholesterol homeostasis in humans and other mammals involves regulation of cholesterol production, bile acid biosynthesis and catabolism of specific serum cholesterol carriers. Important serum cholesterol carriers are called LDL (low density lipoprotein) particles. The LDL receptor has been reported to facilitate internalization of the LDL particle into those cells in need of cholesterol. See e.g., Brown, M. S. and Goldstein, J. L. (1986) Science 232: 34-47; and Goldstein, J. L. and Brown, (1986) Nature, 348: 425; and references cited therein. The LDL receptor has been disclosed as impacting serum cholesterol levels in humans. For example, there has been recognition that cells with enough cholesterol do not make sufficient LDL receptors, thereby reducing or even blocking uptake of cholesterol by the cell. In this instance, serum cholesterol levels rise substantially, which can contribute to the development or severity of disease. Conversely, cells in need of cholesterol often have capacity to make more LDL receptors, thereby facilitating a decrease in serum cholesterol. Accordingly, there has been specific attention focused on regulating the LDL receptor as one therapeutic approach for stabilizing or reducing serum cholesterol levels in human patients.

Accordingly, the peptide according to the invention inhibits the cholesterol biosynthesis pathway by either binding directly and reducing the activity of HMG-CoA enzyme and/or the phosphomevalonate kinase, or by binding to other enzymes at any possible step of the cholesterol biosynthesis pathway. A consequence of the reduction of the cholesterol biosynthesis is the reduction of serum cholesterol.

The peptide according to the invention, in particular the peptide JCH2, is capable of lowering the HMGCoA reductase activity at much lower concentrations than the known inhibitors, Fluvastatin, Mevastatin, Atorvastatin and Simvastatin, while exhibiting some beneficial effects which are different from that of the commonly used statins. Microarray analysis have also shown that besides reducing the reductase mRNA, the compound is also capable of lowering the mevalonate, the precursor for cholesterol biosynthesis The microarray data can be seen in Table 1. The RNA was isolated from HepG2 cell treated with the inhibitors and statins that shows inhibitory activity on HMGCoA reductase assay. The global gene expression (DNA microarray) analysis was carried out on human gene chips (HG_U133A). The data displayed 67 genes that are either up- or down-regulated, upon treatment with venom component. Genes showing two-fold signal to log ratio in expression from two independent experiments were scored as significant.

TABLE 1
Microarray Analysis

| Gene name | Simvastatin | JC | H2 |
|---|---|---|---|
| 1. Cholesterol metabolism | NC | −1.7 | NC |
| 203504_s_at ATP-binding cassette, sub-family A (ABC1), | | | |
| member 1 | | | |
| 2. Calcium channel | −1.6 | −1.9 | NC |
| 214933_at Calcium channel, voltage-dependent, | | | |
| P/Q type, alpha 1A subunit | | | |
| 3. Intracellular Signalling | +2.1 | +1.8 | NC |
| 200605_s_at Protein kinase, | | | |
| cAMP-dependent regulatory, type 1, alpha | | | |
| 4. Small Molecule Transport | +1.8 | +2.3 | +2.5 |
| 201971_s_at ATPase, H+ transporting, | | | |
| lysosomal 70 kDa, V1 subunit A, isoform 1 | | | |
| 5. Cell surface receptor linked signal transduction | NC | +2.0 | +1.7 |
| 211000_s_at Interleukin 6 signal transducer | | | |
| (gp130, oncostatin M receptor) | | | |
| 6. Signal transduction | +2.0 | NC | NC |
| 217941_s_at erbb2 interacting protein (ERBIN) | | | |
| 7. Small Molecule Transport | | | |
| 207408_at Organic cationic transporter-like 4 | NC | −1.4 | NC |
| 205421_at Solute carrier family | +3.5 | NC | +2.6 |
| (extraneuronal monoamine transporter), member 3 | | | |
| 8. Endothelial Recticulum | +2.0 | +1.6 | NC |
| 207791_s_at RAB1A, member RAS oncogene family | | | |
| 9. Cell-cell matrix adhesion | | | |
| 215177_s_at Integrin, alpha 6 | +3.6 | — | — |
| 214021_x_at Integrin, beta 5 | +3.7 | NC | — |
| 10. 208995_s_at Peptidyl-prolyl isomerase G (cyclophilin G) | +2.2 | — | +2.9 |
| 11. Small Molecule Transport | +5.7 | — | — |
| 221487_s_at Endosulfine alpha | | | |
| 12. Mitochondrial | +2.1 | NC | — |
| 200769_s_at Methionine adenosyltransferase II, alpha | | | |
| 13. Smooth Muscle Contraction | +2.1 | NC | NC |
| 201617_x_at Caldesmon 1 | | | |
| 14. Carbohydrate metabolism | MI/+2.0 | NC | NC |
| 202069_s_at Isocitrate dehydrogenase 3 (NAD+) alpha | | | |
| 15. Chromosome condensation/segregation | +5.8 | NC | NC |
| 202131_s_at SudD suppressor of bimD6 homolog | | | |
| (A. nidulans) | | | |
| 16. Transcription | | | |
| 202817_s_at Synovial sarcoma translocation, chromosome | NC | NC | +2.5 |
| 18218129_s_at Nuclear transcription factor Y, beta | +2.3 | — | — |
| 17. Apoptosis | NC | NC | — |
| 203265_s_at Mitogen-activated protein kinase kinase 4 | | | |
| 18. Oncogenesis | | | |
| 204394_at Prostate cancer overexpressed gene 1 | NC | −1.5 | NC |
| 208456_s_at Related RAS viral (r-ras) oncogene homolog 2 | +2.0 | MI/+2.4 | NC |
| 211450_s_at MutS homolog 6 (E. coli) | +2.1 | NC | NC |
| 19. Ossification | −2.2 | NC | NC |
| 208410_x_at Amelogenin (X chromosome, amelogenesis | | | |
| imperfecta 1) | | | |
| 20. GPCRs, Class B Secretin-like | NC/−2.0 | A/−2.3 | — |
| 208593_x_at Corticotropin releasing hormone receptor 1 | | | |
| 21. Heat shock response | +5.6 | — | — |
| 208744_x_at Heat shock 105 kD | | | |
| 22. mRNA splicing | | | |
| 212885_at M-phase phosphoprotein 10 | MI/+2.6 | NC | NC |
| (U3 small nucleolar ribonucleoprotein) | | | |
| 222026_at RNA binding motif protein 3 | NC | NC | +1.7 |
| 23. Ubiquitin-dependent protein degradation | MI/+1.5 | NC | — |
| 209943_at F-box and leucine-rich repeat protein 4 | | | |
| 24. Cell surface receptor linked signal transduction | NC | NC | NC |
| 211339_s_at IL2-inducible T-cell kinase | | | |
| 25. Cell shape and cell size control | NC | NC | NC |
| 214925_s_at Spectrin, alpha, non-erythrocytic 1 (alpha- | | | |
| fodrin) | | | |
| 26. Others | | | |
| 201151_s_at Muscleblind-like (Drosophila) | +2.3 | NC | NC |
| 202375_at SEC24 related gene family, member D | +1.5 | NC | NC |
| (S. cerevisiae) | | | |
| 202840_at TAF15 RNA polymerase II, | MD/−1.5 | −2.3 | NC |
| TATA box binding protein (TBP)-associated factor, 68 kDa | | | |
| 205305_at Fibrinogen-like 1 | NC | −2.6 | NC |
| 206826_at Peripheral myelin protein 2 | NC | — | +2.7 |
| 210941_at BH-protocadherin (brain-heart) | — | NC | +3.4 |
| 211521_s_at Pleckstrin homology, Sec7 and coiled/coil | — | — | — |
| domains 4 | | | |
| 213297_at Nucleolar protein family A, member 2 | NC | NC | NC |
| (H/ACA small nucleolar RNPs) | | | |
| 213534_s_at PAS domain containing serine/threonine kinase | NC | NC | +2.1 |
| 215743_at Ribonuclease P (38 kD) | NC | NC | +2.1 |
| 215954_s_at NY-REN-24 antigen | +2.8 | NC | — |
| 217383_at Phosphoglycerate kinase {alternatively spliced} | NC | −1.2 | NC |
| [Human, phosphoglycerate kinase deficient | | | |
| patient | | | |
| with episodes of muscl, mRNA Partial Mutant, | | | |
| 307 nt] | | | |
| 221618_s_at TAF9-like RNA polymerase II, | +2.2 | — | — |
| TATA box binding protein (TBP)-associated factor, 31 kDa | | | |
| 222119_s_at Vitiligo-associated protein VIT-1 | +1.6 | NC | NC |

(+: increase in expression, −: decrease in expression, NC: no change in gene expression compared to untreated control).

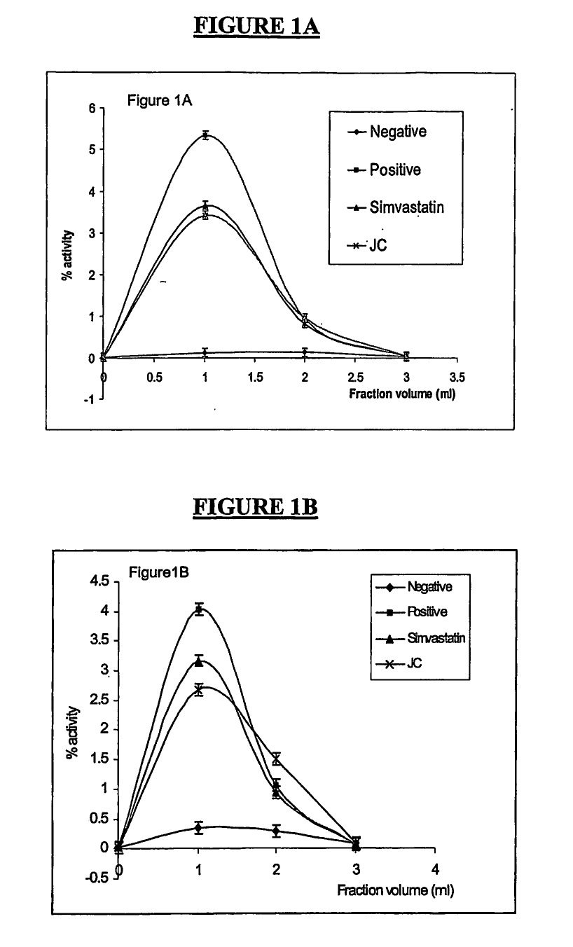

In particular, JCH2 has proved to be a potent inhibitor of HMGCoA reductase in comparison to statins, where the percentage of inhibition of 0.6 μM JCH2 is similar to that of 10 μM statins (FIGS. 1A and 1B, and Table 2). Further analysis (gene expression and protein profiling) has shown that the JCH2 brings about the inhibitory effect possibly via a different pathway than for the existing statins. Both the microarray and the protein profiling data are shown in Table 1 and Table 3. As for the protein profiling data, the profiles as shown in Table 3 were obtained by surface-enhanced laser absorption ionization-time of flight mass spectro-photometry (SELDI-TOF) on treated and control samples using the H4 (hydrophobic) and the NP20 (hydrophilic) chips. Comparison of the Simvastatin treated HepG2 cell protein profile with that of JC or JCH2 treated cells changes shows the specific proteins that are either enhanced or repressed upon treatment with the venom component.

According to a first aspect, the invention provides an isolated nucleic acid molecule selected from the group consisting of:
(a) nucleic acid molecules comprising the nucleotide sequence of SEQ ID NO:1;
(b) nucleic acid molecules encoding a peptide having the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol;
(c) nucleic acid molecules which hybridize to a nucleic acid molecule complementary to the nucleic acid molecule of (a), or (b) or fragment thereof, and which encode a peptide having the function of HMGCoA reductase inhibitor, phosphomevalonate inhibitor, reducing the accumulation of cholesterol in the cholesterol biosynthesis pathway and/or reducing the level of serum cholesterol;
(d) nucleic acid molecules comprising a nucleotide sequence having at least 40% identity with the sequence of SEQ ID NO:1 or a fragment thereof and which encode a peptide having the function of HMGCoA reductase inhibitor, phosphomevalonate inhibitor, reducing the accumulation of cholesterol in the cholesterol biosynthesis pathway and/or reducing the level of serum cholesterol.

The present invention provides a peptide (protein) of SEQ ID NO:2 that inhibits the HMGCoA reductase activity. As used herein, the term variant, derivative, or fragment of the peptide is defined by its ability to inhibit HMGCoA reductase, phosphomevalonate kinase activity, reducing the accumulation of cholesterol in the cholesterol biosynthesis pathway and/or reducing the level of serum cholesterol. With reference to the HMGCoA reductase activity, it can be measured by the assay described in the examples or by other assays available in the art. Accordingly, the present invention also provides a nucleic acid molecule (a), having the sequence of SEQ ID NO:1, and nucleic acid molecules (b), (c) and/or (d), as indicated above, that encodes the peptide in SEQ ID NO:2, or a variant, derivative and/or fragment thereof.

A nucleic acid according to the invention may be a single or double stranded oligonucleotide or polypeptide, or can also be single or double stranded genomic DNA, cDNA, RNA, mRNA. In the case in which a nucleic acid molecule is transcribed and translated to produce a functional peptide, defined in this case as a peptide that retains HMGCoA reductase, phosphomevalonate kinase inhibitory activity, reducing the accumulation of cholesterol in the cholesterol biosynthesis pathway and/or reducing the level of serum cholesterol, one of skill in the art recognizes that because of codon degeneracy a number of different nucleic acids encode the same peptide. In addition, the invention includes those peptides, referred to as variants or derivatives of the peptide in SEQ ID NO:2, that have amino acid sequence substantially identical with the peptide in SEQ ID NO:2 and inhibit HMGCoA reductase, phosphomevalonate kinase activity, reducing the accumulation of cholesterol in the cholesterol biosynthesis pathway and/or reducing the level of serum cholesterol. Variants and/or derivatives include peptides with conserved amino acid changes in the sequence of SEQ ID NO:2. Conserved amino acid substitutions are those changes that can be made without altering the function of the native peptide in SEQ ID NO:2.

In particular, variants of the peptide in SEQ ID NO:2, may be defined as those peptides that contain amino acid substitutions, wherein an amino acid can be replaced with another amino acid without altering the activity of the peptide. These amino acids may or may not be conserved across species and may or may not be essential to the activity inhibitory function of peptide.

In particular, as non-limiting examples, a variant, derivative, and/or a fragment of the peptide of SEQ ID NO:2 are the amino acid sequences SEQ ID NOS:3-13, reported in Table 2. Accordingly, the nucleic acid molecule (b) may be a nucleic acid molecule encoding at least a peptide having the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or SEQ ID NO:13. These sequences and their ability in reducing HMG-CoA reductase activity are reported in Table 2.

Further, homologues of the peptide in SEQ ID NO:2 of the invention are peptides that are “substantially identical” to the peptide in SEQ ID NO:2, retain the inhibitory activity and are found in or isolated from different species of animals than the peptide in the present invention, which is human in origin.

Homologues of the peptide in SEQ ID NO:2 are defined by their ability to inhibit the HMGCoA reductase, phosphomevalonate kinase activity, reduce the accumulation of cholesterol in the cholesterol biosynthesis pathway and/or reduce the level of serum cholesterol in the progression of cholesterol biosynthetic pathway or their by-products. The critical amino acids in a protein or peptide are those amino acids that are important for the function of the protein or peptide. These amino acids are often conserved across different species and organisms. It is well known to one skilled in the art that conserved amino acids are usually identical across species and organisms and if not, are very similar in their chemical properties. Homologues of the peptide in SEQ ID NO:2 are those peptides that are found in other species and organisms that inhibit HMGCoA reductase, phosphomevalonate kinase activity, reduce the accumulation of cholesterol in the cholesterol biosynthesis pathway and/or reduce the level of serum cholesterol. Homologues of the peptide in SEQ ID NO:2 will contain the conserved amino acids that are critical to the function of the peptide. These amino acids are either identical to the corresponding amino acids in SEQ ID NO:2 or have very similar chemical properties.

Accordingly, the nucleic acid molecule (d) may be a nucleic acid molecule comprising a nucleotide sequence having at least 40%, 50%, 60%, 70%, 80% or 90% identity with the sequence of SEQ ID NO:1 or a fragment thereof and which encodes a peptide having the function of HMGCoA reductase inhibitor, phosphomevalonate inhibitor, reducing the accumulation of cholesterol in the cholesterol biosynthesis pathway and/or reducing the level of serum cholesterol. In particular, the nucleic acid molecule (d) has at least 70% identity with the sequence of SEQ ID NO:1 or a fragment thereof, more in particular at least 80%, preferably at least 90%.

Further homologs showing inhibition on HMGCoA reductase activity, phosphomevalonate inhibitor, reducing the accumulation of cholesterol in the cholesterol biosynthesis pathway and/or reducing the level of serum cholesterol, can be obtained from venom of any known species. For example, homologs obtained from the same venom of Buthus martensii Karsch, and showing inhibition on HMGCoA reductase activity, phosphomevalonate inhibitor, reducing the accumulation of cholesterol in the cholesterol biosynthesis pathway and/or reducing the level of serum cholesterol, are reported in Table 2. The molecular weights (masses) of the obtained homologs are the following: 16790 Da (JC1-P1); 16791 Da (JC2-P1) and 17211 Da (JC3-P5) (see Table 2).

With reference to the nucleic acid molecules (c), a nucleic acid molecule is “hybridisable” to another nucleic acid molecule (in the present case, a nucleic acid molecule complementary to the nucleic acid molecule of (a) or (b) or a fragment thereof), when a single-stranded form of the nucleic acid molecule can anneal to the other nucleic acid molecule under appropriate conditions of temperature and solution ionic strength (Sambrook and Russell, Molecular Cloning, A Laboratory Manual, Cold Spring Harbour Laboratory Press, 2001). The conditions of temperature and ionic strength determine the “stringency” of the hybridisation. Hybridisation requires the two nucleic acids to contain complementary sequences. Depending on the stringency of the hybridisation, mismatches between bases are possible. The appropriate stringency for hybridising nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridisation decreases in the following order RNA:RNA, DNA:RNA, DNA:DNA. For hybrids of greater than 100 nucleotides in length, equations for calculating Tm have been derived (Sambrook and Russell, 2001, as above). For hybridisation with shorter nucleic acids, i.e. oligonucleotides, the position of mismatches becomes more important, and the length of the oligonucleotide determines its specificity (Sambrook and Russell, 2001, as above). Preferably a minimum length for a hybridisable nucleic acid is at least about 10 nucleotides; more preferably at least about 15 nucleotides; most preferably the length is at least about 18 nucleotides.

The invention also provides a vector comprising at least one of the nucleic acid molecules (a), (b), (c), (d). In particular, the vector may comprise at least two from the nucleic acid molecules (a), (b), (c) and (d) fused together. The vector may further comprise a regulatory nucleic acid sequence linked to the nucleic acid molecule encoding the polypeptide. The regulatory nucleic acid may be a prokaryotic or eukaryotic promoter.

The invention further provides a host cell comprising the vector. The host cell may be present in the form of a cell culture. The host cell may be a prokaryotic or eukaryotic cell.

In particular, the host cell is cultured to express a peptide having the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol. The host cell may also express at least a peptide having the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or SEQ ID NO:13.

Methods for the preparation of a vector comprising a nucleic acid molecule according to the invention, preferably linked to a promoter, and cultivation of the host cell comprising the vector can be carried out according to standard techniques. For example, as indicated or described in Sambrook and Russel, Molecular Cloning, Cold Spring Harbour, 2002.

According to another aspect, the invention provides an isolated peptide comprising the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol.

Variants, derivatives, homologues and/or fragments of the peptide having the amino acid sequence of SEQ ID NO:2 are as above described.

In particular, according to a non-limiting list, the peptide of the invention may comprise at least one of the amino acid sequences of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13.

The peptide of the invention may also be a fused peptide and comprise at least one peptide having the sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or SEQ ID NO:13.

The peptide of the invention may be isolated and/or purified from biological material, expressed from recombinant DNA, and/or prepared by chemical synthesis.

In particular, the peptide may be isolated and/or purified from a human or non-human animal species.

According to another aspect, the peptide of the invention is isolated and/or purified from the venom. Any suitable venom from any animal comprising venom can be used. The peptide can be isolated and/or purified according to any standard technology known in the art. In particular, the peptide may be obtained from steps comprising:
obtaining crude venom;
carrying out gel filtration; and
performing reverse-phase high performance liquid chromatography.

However, the method of isolating and/or obtaining the peptide of the invention may be any other method known in the art.

According to another aspect, the peptide of the invention is isolated and/or purified to homogeneity from the venom of Buthus martensii Karsch, which is a Chinese scorpion, however, venom from other species may also be used. Accordingly, in particular, the peptide of the invention is isolated and/or purified from the step comprising:
obtaining crude venom of a Buthus martensii Karsch;
carrying out gel filtration;
performing reverse-phase high performance liquid chromatography (RPHPLC).

The peptide isolated form the crude venom of Buthus martensii Karsch is indicated as JCH2 protein or peptide.

In particular, the gel filtration step may be a sequential gel filtration. However, the method for isolation and/or purification of a peptide according to the invention, in particular the peptide JCH2 is not limited to that exemplified herein. Any suitable method known in the art may also be used.

The protein JCH2 showed higher inhibitory activity than the Simvastatin, Mevastatin, Atorvastatin and Fluvastatin when tested for HMGCoA reductase activity on HepG2 cell purified HMGCoA reductase as well as enzymes purified from the rat liver microsomes (FIGS. 1A and 1B). The protein sequence of JCH2 is hereafter referred to as SEQ ID NO:2. The protein has been purified to homogeneity and characterized. The cDNAs encoding JCH2 (SEQ ID NO:1) have been cloned from the venom gland (telson) mRNA. Series of synthetic peptides (SEQ ID NOS:3-13) have also been made and their inhibitory properties were tested.

According to another aspect, the invention provides a pharmaceutical preparation comprising the peptide of the invention, in particular the peptide JCH2 or a homologue, variant, derivative or fragment thereof. The pharmaceutically preparation optionally comprises a pharmaceutically acceptable carrier, diluent, excipient or a combination thereof. The invention encompasses the preparation and use of pharmaceutical compositions comprising the peptide of the invention, in particular the peptide JCH2 or a homologue, variant, derivative or fragment as an active ingredient. Such a pharmaceutical composition may consist of the active ingredient alone, in a form suitable for administration to a subject, or alternatively the pharmaceutical composition may comprise the active ingredient and one or more pharmaceutically acceptable carrier, excipient and/or diluent.

The pharmaceutically preparation may be in the form of oral, parenteral, injection, topical, and/or implant preparation.

Pharmaceutical compositions of peptides or fragments, variants, derivatives and/or homologues of the peptide may be used to inhibit the HMGCoA reductase and/or phosphomevalonate kinase activity, for example, in the treatment or prophylaxis of disorders characterized by the accumulation of cholesterol or its by-product (bile acid) or related lipid derived products (such as HDL, LDL, VLDL etc) such as, for example, hypertension, atherosclerosis, stroke, as well as neurovascular and cardiovascular disorders or in cholesterol independent or pleiotropic effects which could include atherosclerotic stabilization, amelioration of endothelial dysfunction, improved coronary artery compliance and prevention of plaque rupture and thrombus formation.

Compositions of the invention are provided to an animal by any suitable means, directly (e.g., locally, as by injection, implantation or topical administration to a tissue locus) or systemically (e.g., parenterally or orally). Where the compositions of the invention are to be provided parenterally, such as by intravenous, subcutaneous, opthalmic, intraperitoneal, intramuscular, buccal, rectal, vaginal, intraorbital, intracerebral, intracranial, intraspinal, intraventricular, intrathecal, intracistemal, intracapsular, intranasal or by aerosol administration, the composition preferably comprises part of an aqueous or physiologically compatible fluid suspension or solution.

According to another aspect, the invention includes methods for modulating and particularly reducing serum cholesterol level in a mammal. In this embodiment, the methods generally include administering to the mammal a therapeutically effective amount of at least one and typically one of peptide comprising the amino acid sequence of SEQ ID NO: 2, SEQ ID NO:3, SEQ ID NO:4, SEQ ID NO:5, SEQ ID NO:6, SEQ ID NO:7, SEQ ID NO:8, SEQ ID NO:9, SEQ ID NO10, SEQ ID NO:11, SEQ ID NO:12 and SEQ ID NO:13. Further, the method of the invention may also comprise administering a pharmaceutical composition according to the invention.

Cholesterol is generally understood to be essential for normal growth and viability of most higher organisms. Too much serum cholesterol has been correlated with life threatening lipid-related diseases including hyperlipoproteinemia, stoke, coronary heart disease, and especially atherosclerosis and related conditions. See generally Stryer, L. (1988) in Biochemistry, 3.suprd Ed. W. H. Freeman and Co. New York, pp. 547-574; and Brown, M. S. and Goldstein, J. L. (1993) in The Pharmacological Basis of Therapeutics (8 supth Ed.) Gilman, A. G. et al. eds. McGraw-Hill/New York, pp. 874-896.

According to another aspect, the invention provides a method of treatment or prophylaxis of disorders characterised by the accumulation of cholesterol, its by-product and/or related lipid derived products, comprising administering to a subject in need at least one peptide comprising the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol. The method may also comprise administering at least one peptide or a fused peptide comprising the amino acid sequence of at least one of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13. Further, the method of the invention may also comprise administering a pharmaceutical composition according to the invention.

The by-product comprises bile acid. The related lipid derived products comprises HDL, LDL and/or VLDL.

The disorders comprise hypertension, atherosclerosis, stroke, neurovascular and/or cardiovascular disorders.

The peptide or the pharmaceutical preparation may be administered locally, by injection, implantation, topical administration to a tissue locus, parenterally and/or orally. Other forms of administration known in the art and suitable for the purposes of the present method are also within the scope of the present invention.

Apart from their lipid lowering actions, a direct cholesterol dependent activity, statins are proposed to have other important cholesterol-independent or pleiotropic effects including atherosclerotic stabilization, amelioration of endothelial dysfunction, improved coronary artery compliance and prevention of plaque rupture and thrombus formation (Takemoto and Liao 2001, Arterioscier Thromb Vasc Biol 2001 November;21(11):1712-9; Liao, 2002, J Clin Invest 2002 August;110(3):285-8; Vaughan, 2003, Am J Cardiol 2003 Feburary 20;91 (4A):23B-29B).

Accordingly, the invention also provides a method of treatment for cholesterol independent and plelotropic conditions comprising administering to a subject at least one peptide comprising the amino acid sequence of SEQ ID NO: 2. The method may also comprise administering at least one peptide or a fused peptide comprising the amino acid sequence of at least one of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13.

The cholesterol independent and plelotropic condition comprise, but are not limited to, atherosclerotic stabilization, amelioration of endothelial dysfunction, improved coronary artery compliance, prevention of plaque rupture, and/or thrombus formation.

According to another aspect of the present invention is a use of at least one peptide comprising the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol, for the preparation of a medicament for the treatment or prophylaxis of disorders characterised by the accumulation of cholesterol, its by-product and/or related lipid derived products.

In particular, the by-product comprises bile acid and related lipid derived products comprises HDL, LDL and/or VLDL. Further, the disorders comprise hypertension, atherosclerosis, stroke, neurovascular and/or cardiovascular disorders.

The medicament may comprise at least one peptide or a fused peptide comprising the amino acid sequence of at least one of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13. The medicament further comprises a pharmaceutically acceptable carrier, diluent, excipient or a combination thereof. The medicament may be administered locally, by injection, implantation, topical administration to a tissue locus, parenterally and/or orally.

A further aspect of the present invention is the use of at least one peptide comprising the amino acid sequence of SEQ ID NO: 2 for the preparation of a medicament for the treatment for cholesterol independent and pleiotropic conditions. Cholesterol independent and pleiotropic condition comprise atherosclerotic stabilization, amelioration of endothelial dysfunction, improved coronary artery compliance, prevention of plaque rupture, and/or thrombus formation.

The medicament may also comprise at least one peptide or a fused peptide comprising the amino acid sequence of at least one of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13.

Having now generally described the invention, the same will be more readily understood through reference to the following examples, which are provided by way of illustration, and are not intended to be limiting of the present invention.

### EXAMPLES

### Example 1

Human Hepatoma Cell line-HepG2 Cell Culture and Treatment

The cell stocks of Human Hepatoma cell line-HepG2 were cultured in T-25 flask in Eagle's minimum essential medium (MEM), supplemented with 10% fetal bovine serum, and incubated in a humidified incubator (5% CO₂) at 37° C. At 70-80% confluent, treatment was started with 12 hours incubation in complete media supplemented with delipidated FBS (Gibson et al, J Lipid Res. 1990 March;31(3):515-21) This was followed by 4 hours of incubation in complete media with delipidated FBS as well as the inhibitors, statins and JCH2.

After 4 hours incubation with the inhibitors, the media was discarded and cells were washed with 0.90%saline. The cells were scraped and collected into a 50 ml tube. The cells were centrifuged at 2400 rpm for 5 min at 4 C, and the resulting cell pellet was washed twice with 10 ml saline. The final cell pellet was resuspended in 30 μl lysis buffer and briefly sonicated. The suspension was centrifuged at 14,000 rpm for 15 min and the supernatant containing the enzyme, HMGCoA reductase, was used for the assay. Protein concentration was measured by the Bradford method (Bradford M 1976; Anal. Biochem. 72:248-254).

HMGCoA Reductase Activity Assay

In biosynthesis of cholesterol, HMG-CoA reductase is the key enzyme to convert HMG-CoA to its product mevalonate. In the presence of HCl, the mevalonate is converted into mevalonolactone.
[¹⁴C]HMG-CoA+HMG-CoA reductase→[¹⁴C]mevalonate

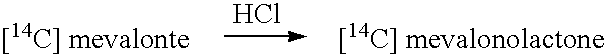

QAE-Sephadex A25 is an anionic-exchange column chromatography (K. K. Ong et al, 1991). It will retain HMG-CoA, which is negatively charge in the column but not mevalonolactone, which will be eluted out by ammonium formate.

The 50 μl assay buffer (100 mM potassium dihydrogen phosphate, 4 mM dithiothreitol, 20 mM glucose-6-phosphate, 20 unit glucose-6-phosphate dehydrogenase, 2.5 mM NADP⁺, 30 μM [14C] HMG-CoA (0.01 μCi) and microsomal protein) was incubated at 37° C. for 1 hour. Reaction was terminated by addition of 25 μl of 2M HCl. After 30 min lactonization of mevalonate to mevalonolactone, 500 μl of 20 mM ammonium formate was added to each sample, spun for 5 min at 14,000 rpm.

Supernatant was applied to the column that is pre-packed with QAE-Sephadex (4 cm), equilibrated with ammonium formate. The first 5 ml fractions was eluted with 20 mM ammonium formate, followed by 5 ml of 0.05M HCl and 10 ml of 0.1M HCl. Radioactivity of the first 5 ml (1 ml fraction) was determined using Beckman LS6500 scintillation counter.

MTT Cell Viability Assay

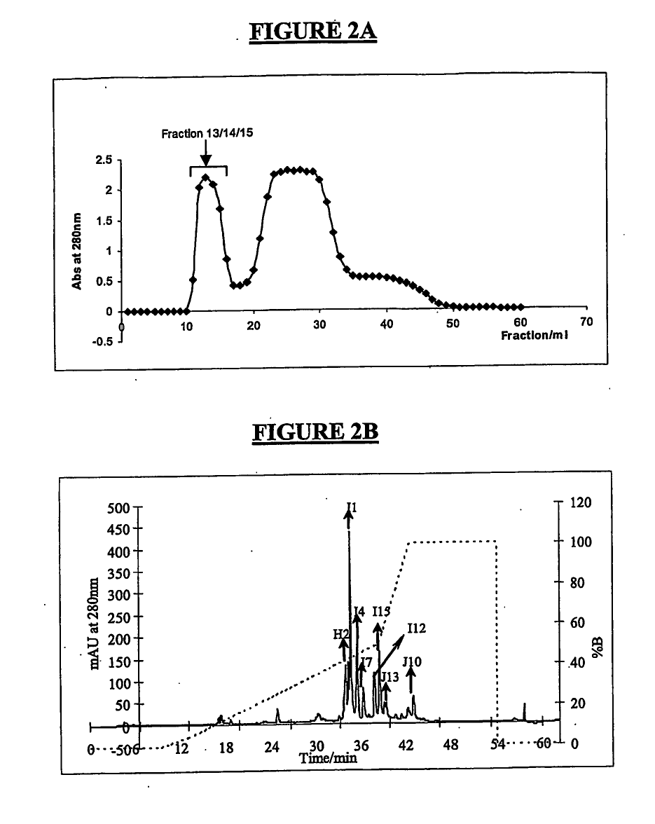

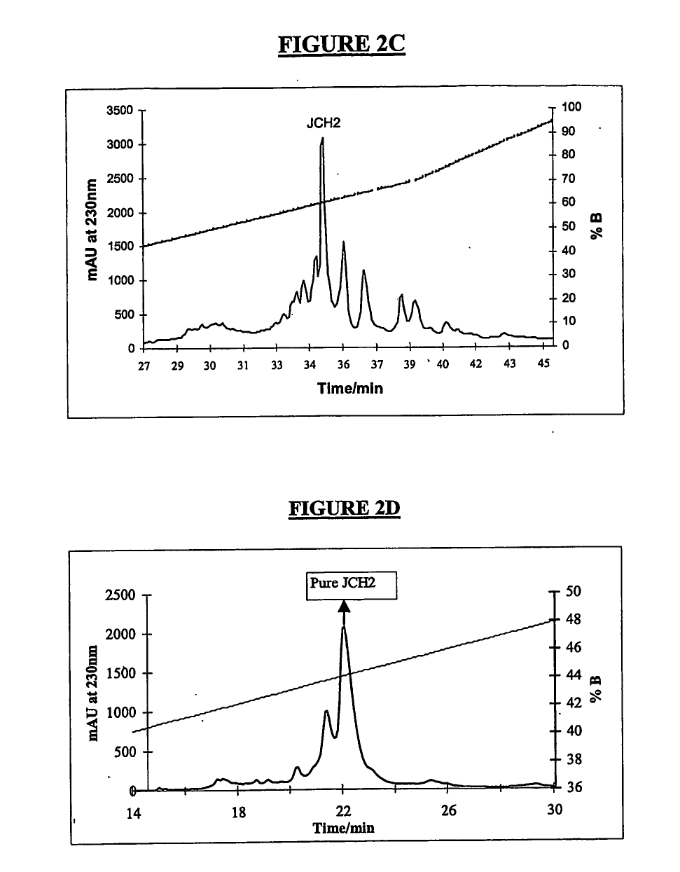

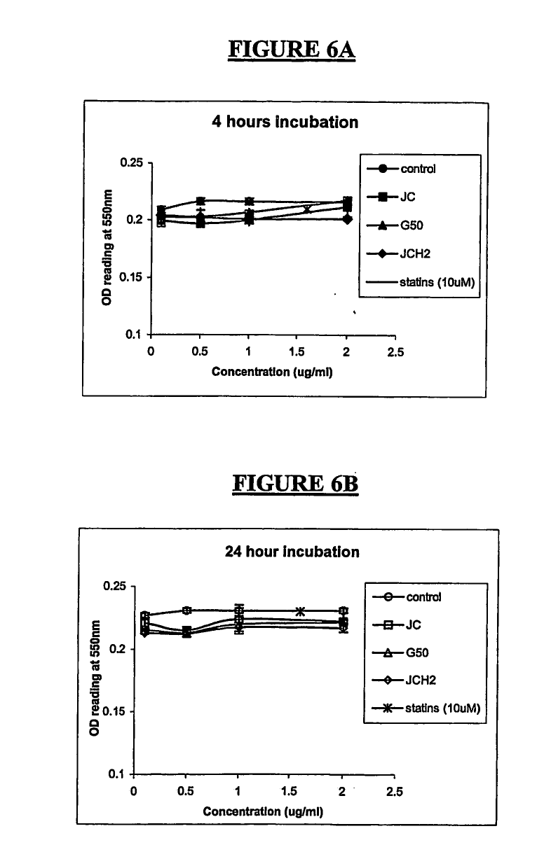

MTT test is a colorimetric assay system (Sigma, USA) which measures the reduction of 3-(4,5-dimethylthiazole-2-yl)-2,5-diphenyl tetrazolium bromide(MTT) into an insoluble formazan product by activity of the mitochondrial enzymes in viable cells. MTT was dissolved in culture medium to a concentration of 0.5 mg/ml and filtered. A 96 well plate was seeded with 5000 cells per well and allowed to grow for 24 hours to 80% confluence. Duplicates were treated with statins and JC fractions. Incubation was carried out for 24 hours. The medium was removed, cells washed twice with 1× saline and then MTT (60 μl) was added. Incubation was carried out for 1 hour and the MTT/medium was removed. The formazan crystals formed were solubilized in 250 ul of dimethylsulfoxide (DMSO). Absorbance was measured in a multiwell scanning spectrophotometer at 550 nm wavelength (FIG. 6). Cell viability (MTT) assay was performed on HepG2 cell treated (a) 4 hours of incubation, (b) 24 hours of incubation, with various concentration of statins (10 uM each), JC (crude venom), G50 (partially purified JC; FIG. 2A) and JCH2 (pure HMGCoA reductase inhibitor from venom; FIG. 2D. The viability (Abs at 550) was then plotted against protein concentration of the inhibitors.

HMGCoA Reductase Activity of Venom

Venom from Chinese scorpion (Buthus martensi Karsch) was tested for HMGCoA reductase activity using Simvastatin, Atoravstatin, Mevastatin and Fluvastatin as a control for inhibition. Protein concentration of 1-100 μg/ml was used. The inhibitory activity was observed for statins as well as for the scorpion venom (FIG. 1A). FIG. 1A shows the results of the HMGCoA reductase assay, using the enzyme isolated from rat hepatocytes, using both simvastatin (10 μM) and crude JC (10 μg). The inhibitors were added to the enzyme assay. FIG. 1B shows the results from the HMGCoA reductase assay for HepG2 cells treated with simvastatin (10 μM) and crude JC (10 μg). The cells were then scraped and sonicated to break the cell for proteins. The cell proteins were used as enzyme source. In both cases (FIGS. 1A and 1B), the positive controls were cells without any treatment while the negative controls were assays without the enzyme.

Fractionation of Buthus martensii Karsch Crude Venom by Gel Filtration

The Chinese scorpion venom was chromatographed on Sephadex G-50 gel filtration using 10 mM Tris-Cl pH 7.6. FIG. 2a shows the elution profile of the crude venom. The HMGCoA reductase inhibition was seen for fractions from peak 1, P1, at 10 μg protein. Based on this result, P1 fractions (13, 14, 15), were subjected to reverse phase HPLC (RP-HPLC) on a C18 column (Jupiter C18, 5 um; 4.6×250 mm). The column was equilibrated with 0.1% TFA in H₂O and the protein was eluted using a step gradient of 0-20% B, followed by 20-70% B and finally 70%-100% B (0.05% TFA in 80% acetonitrile; FIG. 2b). All the fractions from RP-HPLC were assayed for the HMGCOA reductase activity at 10 μg protein. The H2 fraction showing high inhibitory activity was further purified on RP-HPLC (FIG. 2c). The H2 fraction were further purified on Jupiter C4 column and tested for inhibitory property (FIG. 2d).

Cloning of the JCH2 from Buthus martensii Karsch

Total RNA extracted (Chomczynski, P. & Sacchi, N., 1987, Anal. Biochem. 162, 156-159) from the telsons, was reversed transcribed (Armugam, A., et al., 1997, Toxicon 35, 27-37.26). The double stranded cDNA was synthesised and ligated to universal adaptor (AP) on both ends. The adapted cDNAs were used as a source for amplifying specific clones using the gene specific primers forward 1:
5′GA(T/C)AG(T/C)CTNTCNCC(T/C)TGGAA(T/C)GA3′ (SEQ ID NO:14)
   (with the form “(T/C)” it is indicated that in that position the nucleotide can be T or C); and universal adaptor primer as reverse primer, API:
5′ GTMTACGACTCACTATAGGGC 3′ (SEQ ID NO:15).

A similar reaction was carried out using a gene specific reverse primer
5′ATTCCMGG GGAAAGACTATC 3′ (SEQ ID NO:16)
   and the adaptor primer, AP1, as the forward primer. Both the PCR products were sequenced and the cDNA encoding JCH2 was amplified by PCR using the following gene specific primers: forward primer:
5′ GGTACATTTCTAAAAAAAGTTATG 3′ (SEQ ID NO:17)
   and reverse primer:
5′ AATAGCATTGATTAAATGCAAATC 3′ (SEQ ID NO:18).

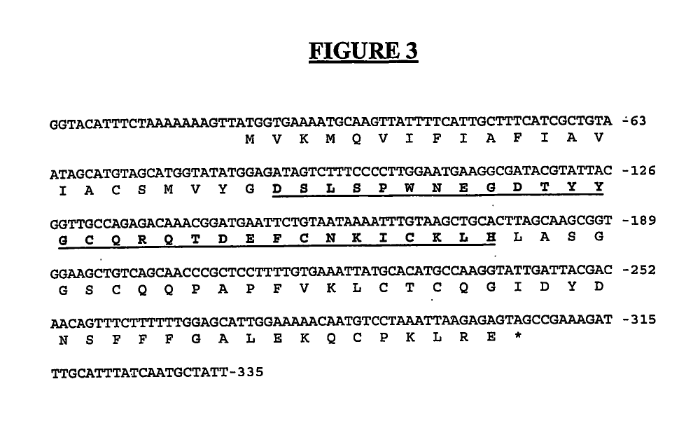

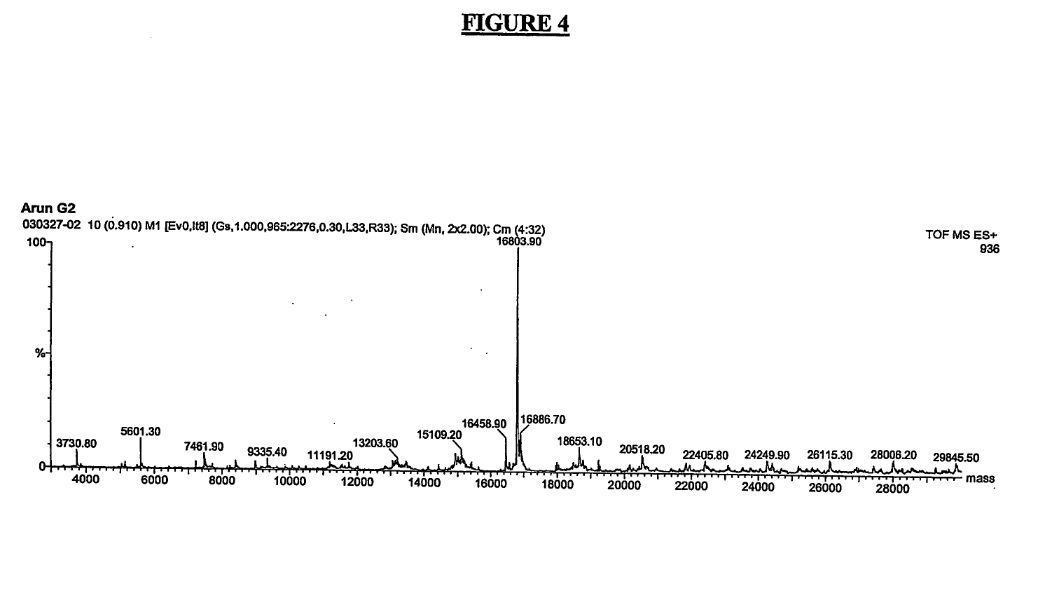

An approximately 400 bp fragment of PCR product was obtained and subcloned. Putative positive clones were subjected to DNA sequencing (Sanger, F., et al., 1977, Proc. Natl. Acad. Sci. USA 74, 5463-5467; Armugam et al, 1997) on an automated DNA sequencer (Model 3100, Applied Biosystems, USA). All clones showed the presence of cDNA encoding JCH2 (FIG. 3). The cDNA encoded for a 22 amino acid signal peptide and 72 amino acid residues of the mature protein (FIG. 3).

The proteins have been found to be negatively charged with pl value (5.03) corresponding to acidic proteins. Interestingly, the mass of 16 kDa (obtained from protein analysis) does not correlate to the mass deduced from cDNA, The mass deduced from the cDNA sequence is 8132 Da while the mass of native protein was determined to be 16803 Da. The JCH2 cDNA, showed that the JCH2 contains 7 Cys residues. Thus, only 3 disulphide bridges have been predicted for this protein. However the extra Cys residue might be involved in forming a covalent bonded dimer, as observed in the mass analysis (16803 Da).

Characterization of JCH2 Protein

The purified native JCH2 was subjected to Mass spectrophotometer analysis. Mass spectrometry analyses were carried out on a Q-TOFF mass spectrophotometer (Micromass UK Ltd). The processing of the mass spectra was performed using the Micromass MassLynx software according to the manufacturer's protocol. The mass was determined to be 16803 Da (FIG. 2e). The N-terminal amino acid sequence of this protein was determined to be —DSLSPWNEGDTYYGCQRQTDEFCNKICKLH—(SEQ ID NO:4) for the first 30 amino acid residues.

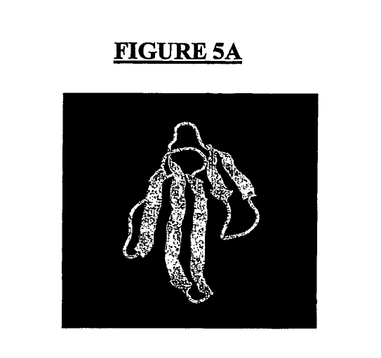

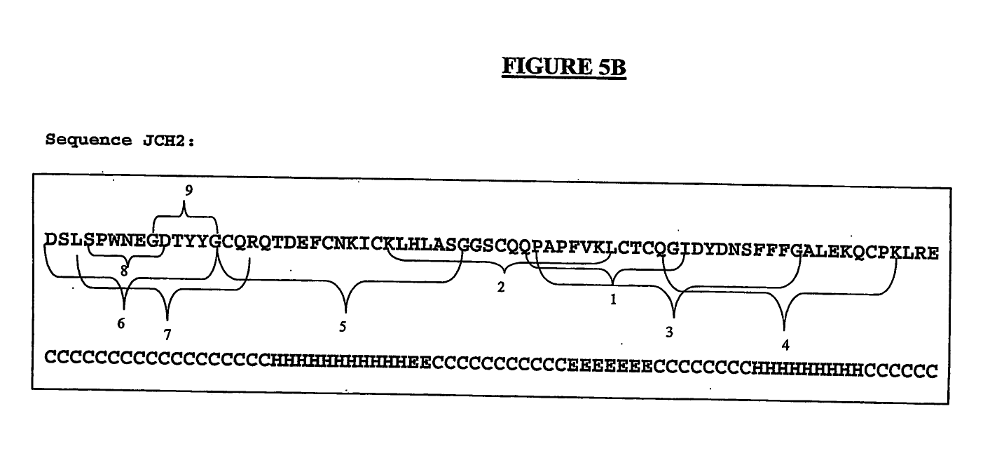

Homology modelling was performed to predict the tertiary structure of JCH2 (FIGS. 5A and 5B). Synthetic peptides were made based on the amino acid sequence of JCH2 and further tested for the HMGCoA reductase inhibitory activity (FIGS. 5A and 5B). All the HMGCoA reductase activity obtained for the protein fractions, statins and the synthetic peptides are presented in Table 2.

TABLE 2

HMGCoA reductase activity of protein, statins and synthetic peptides.
HepG2 cells were treated with native proteins at 10 μg each [crude venom, JCG50
(FIG. 2A), purified JCH2 (FIG. 2D)], statins at 10 uM each (Atorvastatin, Fluvastatin,
imvastatin and Mevastatin) and the synthetic peptides at 10 uM (derived from SEQ ID
NO:3). The peptides and statins were dissolved in either DMSO or sterile water.
Percentage of inhibition was calculated based on positive control (untreated cells):
The sequences of homologs to JCH2 have the followings molecular weights
(masses): 16790Da (JC1-P1); 16791Da (JC2-P1) and 17211Da (JC3-P5).

% of inhibition on
HMG-CoA
reductase activity

| | | |
|---|---|---|
| | Aqueous | DMSO |
| Description/Sequence of protein & peptides | medium | medium |

| | | | |
|---|---|---|---|
| Crude venom | | 21% | — |
| (10 μg) | | | |
| JCG50-P1 | Peak 1 from Sephadex G50 chromatography | 25% | — |
| (10 μg) | | | |
| JCH2 (0.6 μM) | Native protein (RP-HPLC) | 30% | — |
| SEQ ID NO:3 | DSLSPWNEGDTYYGCQRQTDEFCNKICKLHLASG | | |
| | GSCQQPAPFVKLCTCQGIDYDNSFFFGALEKQCPK | | |
| | LRE | | |
| JC1-P1 | Similar mass with JCH2 (16790Da for JC1-P1; 16791Da | 23% | |
| JC2-P1 | for JC2-P1 and 17211Da for JC3-P5) | 25% | |
| JC3-P5 | | 20% | |

| | | | | |
|---|---|---|---|---|
| Peptide 1 | QPAPFVKLCTCQGI | (SEQ ID NO:5) | 20% | 20% |
| Peptide 2 | KLHLASGGSCQQPAPFVKL | (SEQ ID NO:6) | 30% | 7% |
| Peptide 3 | PAPFVKLCTCQGIDYDNSFFFG | (SEQ ID NO:7) | 10% | 15% |
| Peptide 4 | QGIDYDNSFFFGALEKQCPK | (SEQ ID NO:8) | 7% | — |
| Peptide 5 | GCQRQTDEFCNKICKLHLASG | (SEQ ID NO:9) | 2% | — |
| Peptide 6 | DSLSPWNEGDTYYG | (SEQ ID NO:10) | 4% | — |
| Peptide 7 | LSPWNEGDTYYGCQR | (SEQ ID NO:11) | 2% | — |
| Peptide 8 | SPWNEGD | (SEQ ID NO:12) | 1% | — |
| Peptide 9 | GDTYYG | (SEQ ID NO:13) | 3% | — |

| | | | |
|---|---|---|---|
| Atorvastatin | An HMG-CoA reductase inhibitor, reduces total | Insoluble in | 70% |
| (hydrophobic) | cholesterol, LDL and apolipoprotein B. | aqueous | |
| | | medium | |
| Fluvastatin | A synthetic, competitive inhibitor of HMG-CoA | Insoluble in | 74% |
| (hydrophobic) | reductase that acts as anti-hypercholesterolemic | aqueous | |
| | agent, reduces the basal MMP-1 levels in | medium | |
| | culture media of endothelial cells | | |
| Simvastatin | A lipophilic HMG-CoA reductase inhibitor that blocks | 35% | 65% |
| (hydrophilic) | Ras function and inhibits cell proliferation of human | | |
| (10 μM) | smooth muscle cells | | |
| Mevastatin | Compactin: an antibiotic that acts as a potent inhibitor of | 35% | 60% |
| (hydrophilic) | HMG-CoA reductase, suppresses Ras farnesylation. | | |
| | Causes cell cycle arrest in late G1 phase | | |

Note:
Concentrations of all peptides and statins are 10 μM except for JCH2 which is at 0.6 μM (10 μg)

TABLE 3
Protein profiling data of hydrophobic chip, H4 (Table 3A) and
hydrophilic chip, NP20 (Table 3B).

| | Molecular | Treatment | | |
|---|---|---|---|---|
| Protein | weight (kDa) | Sim | JC | JCH2 |

Table 3A

| | | | | |
|---|---|---|---|---|
| ApoC-I | 6.6 | ↑ ↑ | ↓ ↓ | ↓ |
| ApoC-II/III | 8.8 | ↑ | ↓ ↓ | ↑ ↑ |
| ApoC-IV, SAA-1 protein | 11.0 | ↓ | ↑ | ↓ |
| sPLA₂, FABP (aP2) | 14.0 | ↓ | ↑ | ↓ |
| StAR related lipid transfer protein | 23.0 | ↑ | ↓ | ↑ ↑ |
| ApoA-I | 29.0 | ↓ | ↑ | ↑ |
| ApoE | 33.0 | ↓ | ↑ ↑ | ↑ |
| Oxysterol binding protein-related | 55.0 | ↓ | ↓ | ND |
| protein 2, Hepatic lipase/ | | | | |
| Triacylglycerol lipase | | | | |

Table 3B

| | | | | |
|---|---|---|---|---|
| ApoC-I | 6.6 | ↑ ↑ | ↑ | ↑ |
| ApoC-II/III | 8.8 | ↑ | ↑ | ↑ |
| ApoC-IV, SAA-1 protein | 11.0 | ↑ ↑ | ↑ | ↑ |
| sPLA₂, FABP (aP2) | 14.0 | ↑ ↑ | ↑ | ↑ |
| PMKase, ApoM | 21.0 | ↓ | ↓ | ↓ |
| StAR related lipid transfer protein | 23.0 | ↓ | ↓ | ↓ |
| ApoE | 33.0 | ↑ | ↑ | ↑ |

↓: down-regulated,
↑: up-regulated,
ND: not detected

1-40. (canceled)

41. An isolated nucleic acid molecule selected from the group consisting of:
(a) nucleic acid molecules comprising the nucleotide sequence of SEQ ID NO:1;
(b) nucleic acid molecules encoding a peptide having the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol;
(c) nucleic acid molecules which hybridize to a nucleic acid molecule complementary to the nucleic acid molecule of (a), or (b) or fragment thereof, and which encode a peptide having the function of HMGCoA reductase inhibitor, phosphomevalonate inhibitor, reducing the accumulation of cholesterol in the cholesterol biosynthesis pathway and/or reducing the level of serum cholesterol; and
(d) nucleic acid molecules comprising a nucleotide sequence having at least 40% identity with the sequence of SEQ ID NO:1 or a fragment thereof and which encode a peptide having the function of HMGCoA reductase inhibitor, phosphomevalonate inhibitor, reducing the accumulation of cholesterol in the cholesterol biosynthesis pathway and/or reducing the level of serum cholesterol.

42. The nucleic acid molecule of claim 41, wherein the nucleic acid molecule (b) encodes at least a peptide, or fragment thereof, having the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or SEQ ID NO:13.

43. The nucleic acid molecule of claim 41, wherein the nucleic acid molecule (d) comprises a nucleotide sequence having at least 40%, 50%, 60%, 70%, 80% or 90% identity with the sequence of SEQ ID NO:1 or a fragment thereof and which encode a peptide having the function of HMGCoA reductase inhibitor, phosphomevalonate inhibitor, reducing the accumulation of cholesterol in the cholesterol biosynthesis pathway and/or reducing the level of serum cholesterol.

44. The nucleic acid molecule of claim 41, wherein the nucleic acid molecule is a single or double strand polynucleotide, oligonucleotide, genomic DNA, cDNA, RNA, mRNA.

45. An expression vector, comprising at least one of the nucleic acid molecules (a), (b), (c), (d) of claim 41.

46. The expression vector of claim 45, wherein the expression vector further comprises a regulatory nucleic acid sequence.

47. The expression vector of claim 46, wherein the regulatory nucleic acid sequence is linked to the nucleic acid molecule encoding the polypeptide.

48. The expression vector of claim 46, wherein the regulatory nucleic acid sequence is that of a prokaryotic or eukaryotic promoter.

49. The expression vector of claim 45, wherein at least two from the nucleic acid molecules of (a), (b), (c) and (d) are fused together in the vector.

50. A host cell, wherein the host cell comprises the vector of claim 45.

51. The host cell of claim 50, wherein the host cell is in the form of cell culture.

52. The host cell of claim 50, wherein the host cell is a prokaryotic or eukaryotic cell.

53. The host cell of claim 50, wherein the host cell is cultured to express a peptide having the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol.

54. The host cell of claim 50, wherein the host cell expresses at least a peptide, or a fragment thereof, having the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or SEQ ID NO:13.

55. An isolated peptide comprising the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol.

56. The peptide of claim 55, wherein the peptide, or fragment thereof, comprises the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or SEQ ID NO:13.

57. The peptide of claim 55, wherein the peptide is a fused peptide and comprises at least one peptide, or fragment thereof having the sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or SEQ ID NO:13.

58. The peptide of claim 55, wherein the peptide is isolated and/or purified from a human or non-human animal species.

59. The peptide of claim 55, wherein the peptide is isolated and/or purified from the venom.

60. The peptide of claim 59, wherein the venom is from Buthus martensii Karsch.

61. The peptide of claim 55, wherein the peptide is obtained from steps comprising:
obtaining crude venom;
carrying out gel filtration; and
performing reverse-phase high performance liquid chromatography.

62. The peptide of claim 55, wherein the molecular weight of the peptide is 16803 Da, 16790 Da ; 16791 Da or 17211 Da.

63. The peptide of claim 55, wherein the peptide is isolated and/or purified from biological material, expressed from recombinant DNA, and/or prepared by chemical synthesis.

64. An isolated peptide, wherein the peptide has the function of HMGCoA reductase inhibitor, phosphomevalonate inhibitor, reducing the accumulation of cholesterol in the cholesterol biosynthesis pathway and/or reducing the level of serum cholesterol, and wherein the peptide has a molecular weight of 16803 Da, 16790 Da, 16791 Da or 17211 Da.

65. The isolated peptide of claim 64, wherein the peptide is isolated and/or purified from the venom of Buthus martensii Karsch.

66. A pharmaceutical preparation comprising a peptide, wherein the peptide is at least one of:
(a) an isolated peptide comprising the amino acid sequence of SEQ ID NO: 2 or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol; or
(b) an isolated peptide, wherein the peptide has the function of HMGCoA reductase inhibitor, phosphomevalonate inhibitor, reducing the accumulation of cholesterol in the cholesterol biosynthesis pathway and/or reducing the level of serum cholesterol, and wherein the peptide has a molecular weight of 16803 Da, 16790 Da, 16791 Da or 17211 Da.

67. The pharmaceutical preparation of claim 66, wherein the pharmaceutical preparation further comprises a pharmaceutically acceptable carrier, diluent, excipient or a combination thereof.

68. The pharmaceutical preparation of claim 66, wherein the pharmaceutically preparation is in the form of oral, parenteral, injection, topical, and/or implant preparation.

69. A method for the treatment or prophylaxis of disorders characterised by the accumulation of cholesterol, its by-product and/or related lipid derived products, the method comprising a step of administering to a subject in need at least one of:
(a) a peptide comprising the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol; and/or
(b) an isolated peptide, wherein the peptide has the function of HMGCoA reductase inhibitor, phosphomevalonate inhibitor, reducing the accumulation of cholesterol in the cholesterol biosynthesis pathway and/or reducing the level of serum cholesterol, and wherein the peptide has a molecular weight of 16803 Da, 16790 Da, 16791 Da or 17211 Da.

70. The method of claim 69, wherein the peptide is at least one peptide or a fused peptide, or fragment thereof, comprising the amino acid sequence of at least one of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13.

71. The method of claim 69, wherein the by-product comprises bile acid.

72. The method of claim 69, wherein the related lipid derived products comprises HDL, LDL and/or VLDL.

73. The method of claim 69, wherein the disorders comprise hypertension, atherosclerosis, stroke, neurovascular and/or cardiovascular disorders.

74. The method of claim 69, wherein the peptide is administered with a pharmaceutically acceptable carrier, diluent, excipient or a combination thereof.

75. The method of claim 69, wherein the peptide is administered locally, by injection, implantation, topical administration to a tissue locus, parenterally and/or orally.

76. A method for the treatment of cholesterol independent and pleiotropic conditions, the method comprising a step of administering to a subject in need at least one of:
(a) a peptide comprising the amino acid sequence of SEQ ID NO: 2;
(b) an isolated peptide comprising the amino acid sequence of SEQ ID NO: 2, or a variant, derivative and/or fragment thereof having the function of HMGCoA reductase inhibitor, phosphomevalonate inhibitor, reducing the accumulation of cholesterol in the cholesterol biosynthesis pathway and/or reducing the level of serum cholesterol; or
(c) an isolated peptide, wherein the peptide has the function of HMGCoA reductase inhibitor, phosphomevalonate inhibitor, reducing the accumulation of cholesterol in the cholesterol biosynthesis pathway and/or reducing the level of serum cholesterol, and wherein the peptide has a molecular weight of 16803 Da, 16790 Da, 16791 Da or 17211 Da.

77. The method of claim 76, wherein the peptide is at least one peptide or a fused peptide, or fragment thereof, comprising the amino acid sequence of at least one of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:13.

78. The method of claim 76, wherein the cholesterol independent and pleiotropic conditions comprise atherosclerotic stabilization, amelioration of endothelial dysfunction, improved coronary artery compliance, prevention of plaque rupture, and/or thrombus formation.


SEQUENCE LISTING (18 sequences)

SEQ ID NO: 1 - DNA, 335 bp, Buthus martensii Karsch
ggtacatttctaaaaaaagttatggtgaaaatgcaagttattttcattgctttcatcgctgtaatagcatgtagcatggtatatggagatagtctttccccttggaatgaaggcgatacgtattacggttgccagagacaaacggatgaattctgtaataaaatttgtaagctgcacttagcaagcggtggaagctgtcagcaacccgctccttttgtgaaattatgcacatgccaaggtattgattacgacaacagtttcttttttggagcattggaaaaacaatgtcctaaattaagagagtagccgaaagatttgcatttatcaatgctatt

SEQ ID NO: 2 - protein, 94 aa, Buthus martensii Karsch
MVKMQVIFIAFIAVIACSMVYGDSLSPWNEGDTYYGCQRQTDEFCNKICKLHLASGGSCQQPAPFVKLCTCQGIDYDNSFFFGALEKQCPKLRE

SEQ ID NO: 3 - protein, 72 aa, Buthus martensii Karsch
DSLSPWNEGDTYYGCQRQTDEFCNKICKLHLASGGSCQQPAPFVKLCTCQGIDYDNSFFFGALEKQCPKLRE

SEQ ID NO: 4 - protein, 30 aa, Buthus martensii Karsch
DSLSPWNEGDTYYGCQRQTDEFCNKICKLH

SEQ ID NO: 5 - protein, 14 aa, Buthus martensii Karsch
QPAPFVKLCTCQGI

SEQ ID NO: 6 - protein, 19 aa, Buthus martensii Karsch
KLHLASGGSCQQPAPFVKL

SEQ ID NO: 7 - protein, 22 aa, Buthus martensii Karsch
PAPFVKLCTCQGIDYDNSFFFG

SEQ ID NO: 8 - protein, 20 aa, Buthus martensii Karsch
QGIDYDNSFFFGALEKQCPK

SEQ ID NO: 9 - protein, 21 aa, Buthus martensii Karsch
GCQRQTDEFCNKICKLHLASG

SEQ ID NO: 10 - protein, 14 aa, Buthus martensii Karsch
DSLSPWNEGDTYYG

SEQ ID NO: 11 - protein, 15 aa, Buthus martensii Karsch
LSPWNEGDTYYGCQR

SEQ ID NO: 12 - protein, 7 aa, Buthus martensii Karsch
SPWNEGD

SEQ ID NO: 13 - protein, 6 aa, Buthus martensii Karsch
GDTYYG

SEQ ID NO: 14 - DNA, 23 bp, Buthus martensii Karsch
  misc_feature (1)..(23): y = t or c
gayagyctntcnccytggaayga

SEQ ID NO: 15 - DNA, 22 bp, Artificial
  oligonucleotide primer with homology to universal adaptor AP1 wich is ligated onto each end of Buthus martensii Karsch
gtaatacgactcactatagggc

SEQ ID NO: 16 - DNA, 21 bp, Buthus martensii Karsch
attccaaggggaaagactatc

SEQ ID NO: 17 - DNA, 24 bp, Buthus martensii Karsch
ggtacatttctaaaaaaagttatg

SEQ ID NO: 18 - DNA, 24 bp, Buthus martensii Karsch
aatagcattgattaaatgcaaatc